(19) United States
(12) Patent Application Publication
(10) Pub. No.: US 2003/0034267 A1
(43) Pub. Date: Feb. 20, 2003

(54) Case for the protection and the transportation of epidermis or skin grafts cultivated in vitro from a skin graft cut from a donor

(76) Inventors: Michele De Luca, Ardea (IT); Graziella Pellegrini, Ardea (IT)

Correspondence Address: YOUNG & THOMPSON, 745 SOUTH 23RD STREET 2ND FLOOR, ARLINGTON, VA, 22202

(21) Appl. No.: 09/928,445

(22) Filed: Aug. 14, 2001

(51) Int. Cl. A61B 19/02; C12M 3/00

(52) U.S. Cl. 206/438; 435/307.1

(57) ABSTRACT

A case for the protection and transportation of epidermis or skin grafts includes a lid (1) and a tray (2) able to be superimposed and fitted on each other so as to define a closed, essentially flat space (V) able to contain the epidermis or skin graft immersed in a preservation liquid under stretched conditions. Both the lid (1) and tray (2) are provided with a peripheral flange (F) which is able to be heat sealed. Space (V) is small enough to avoid the wrinkling of the skin graft contained therein, even if the case is inclined.

The present finding relates to the skin engineering and epidermis grafts and particularly a case able to receive and to protect epidermis or skin grafts cultivated in vitro both during their storing in the refrigerator and the transportation from the production and/or storing site to the repair site where they will be grafted to the donor patient.

Especially in the surgical therapies for treating patients affected by severe burns, it is known how hard is to find epidermis or skin grafts which are large enough and in such a good state as to allow their easy grafting to the recipient portions of the human body devoid of epidermis or skin. This is also valid in case of interventions on venous ulcer.

Another problem is to find epidermis or skin grafts that do not cause any rejection.

The present techniques of skin engineering have overcome such problems by providing in vitro epidermis or skin cultivation methods starting from an even small epidermis or skin graft of the patient to be treated.

One problem arising in such case, however, is the delicacy of the skin grafts and the difficulty of storage and transportation of such skin grafts cultivated in vitro that usually have a square or rectangular shape.

The present finding seeks to overcome said problems by providing a case which can be easily stacked and capable of protecting and sealing the single epidermis or skin grafts placed in the preservation solution even during any transfer to an incubator, thus avoiding their curling and wrinkling both during the transportation and the storage as well as the escape of gases contained in the preservation solution and the inlet of the atmosphere gases.

A better understanding of the finding will result from the following detailed description with reference to the accompanying drawings that show some preferred embodiments thereof only by way of a not limiting example.

In the drawings:

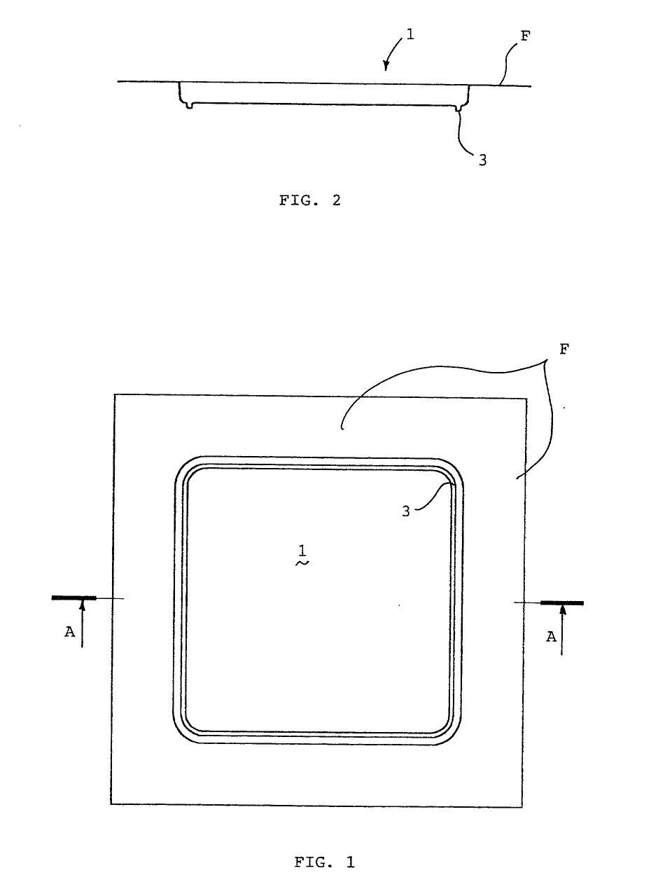

FIG. 1 is a top plan view of the upper portion or lid of the case according to the finding;

FIG. 2 is a cross section of the lid along the plane A-A of FIG. 1;

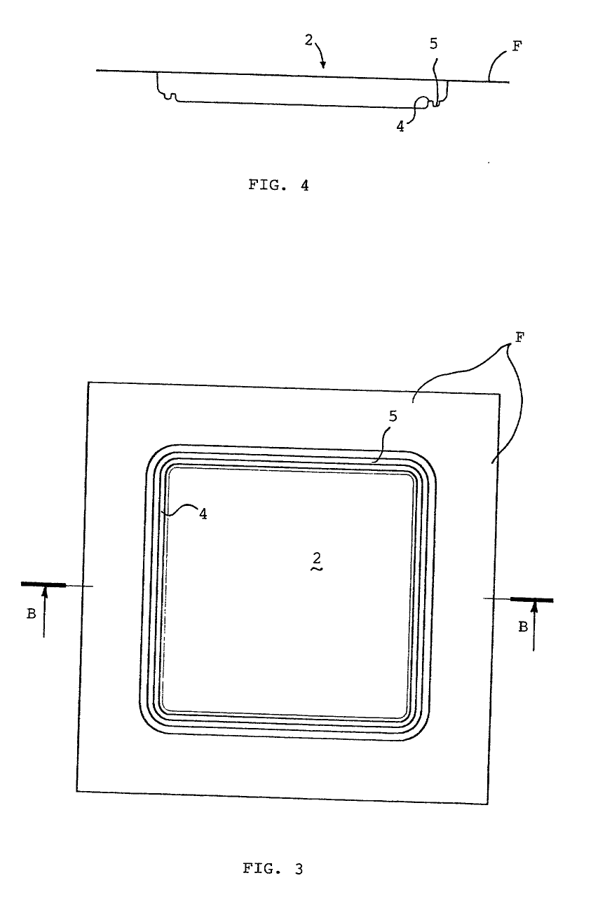

FIG. 3 is a top plan view of the lower portion or tray of the case according to the finding;

FIG. 4 is a cross section of the tray along the plane B-B of FIG. 3;

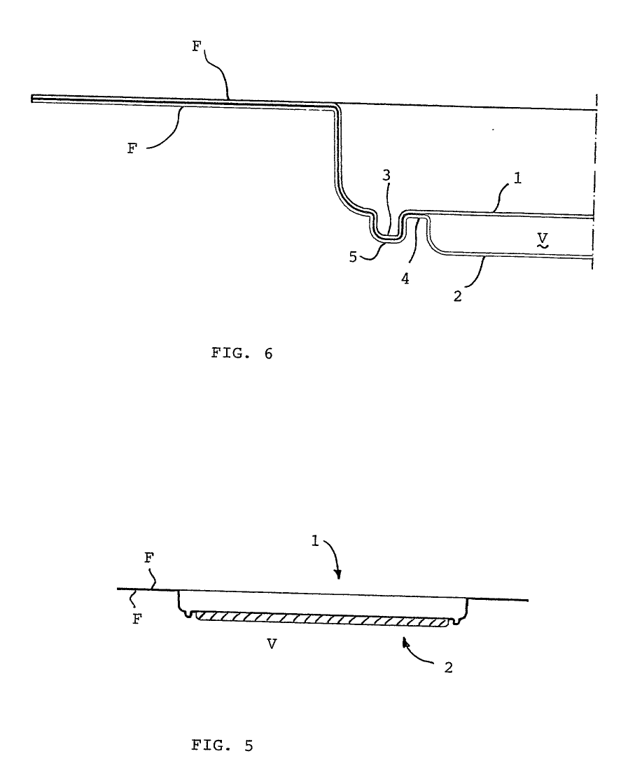

FIG. 5 is a cross section similar to FIGS. 2 and 4 of the superimposed tray and lid in the transportation position;

FIG. 6 is an enlarged detail of the section of FIG. 5;

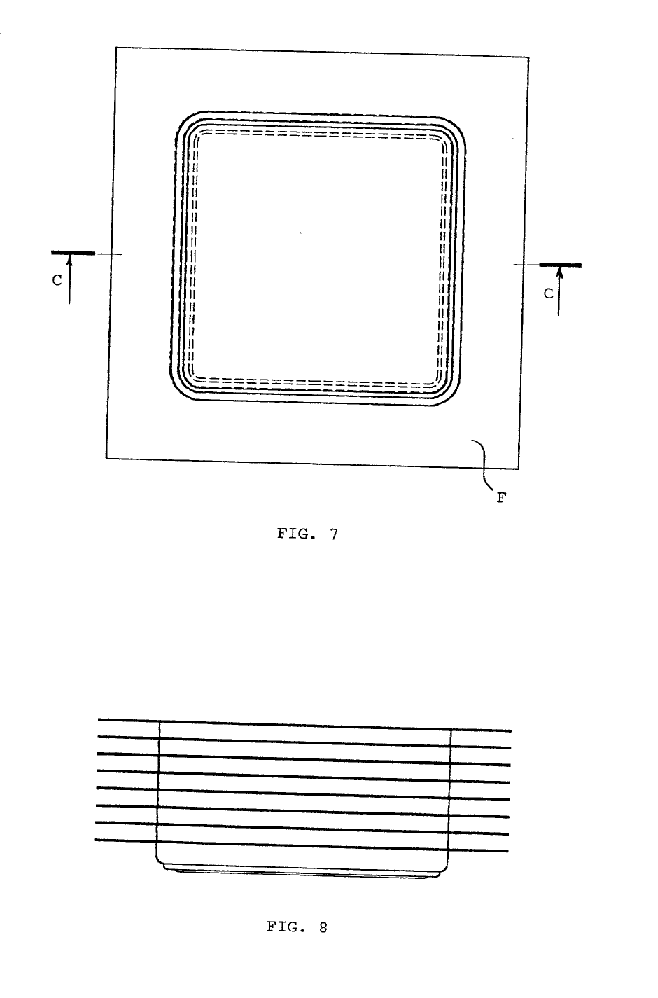

FIG. 7 is a top plan view of several cases stacked on one another according to the finding;

FIG. 8 is a cross section of the stacked cases of FIG. 7 along the plane C-C.

With reference to the figures listed above relating by way of example to a case with essentially square shape, the case consists of a lid 1 and a tray 2 able to be superimposed and fitted on each other so as to define a closed, essentially flat space V (dashed in FIG. 5) able to contain the epidermis or skin graft cultivated in vitro and immersed in a culture liquid under stretched conditions.

According to the present finding, said space is small enough to avoid the wrinkling of the skin graft contained therein, even if the case is inclined.

With particular reference to FIG. 1 and 3, the lid has an essentially flat central portion which is lowered with respect to its peripheral edge and is provided with a continues peripheral little channel which projects from the underlying surface so as to form a corresponding peripheral lower rib 3 projecting from the flat surface of the lowered portion of the lid.

Tray 2 shown in FIGS. 3 and 4 has a flat bottom surrounded and delimited by a continuous peripheral rib 4 which is raised with respect to the bottom and outside which there is provided a small channel 5 running parallel to said rib 4 and able to fit such lower rib 3 of lid 1.

In the described preferred embodiment the bottom of tray 2 is deeper than the peripheral small channel 5.

As shown in FIGS. 5 and 6, space V able to receive the cultivated epidermis or skin graft and the culture liquid necessary to its preservation is defined by the empty space delimited at the lower side by the central portion of tray 2 and at the upper side by the flat bottom of lid 1. Such lid 1 and such tray 2 are superimposed by fitting rib 3 into small channel 5, as described above.

According to a peculiar characteristic of the finding, the case is made of a chemically and biologically inert material which is impermeable to liquids and gases. The liquid and gas sealing is advantageously ensured by that the peripheral portions of lid 1 and tray 2 are shaped such as to provide perfectly conjugated surfaces ensuring the sealing by fitting rib 3 into small channel 5, as described above.

Once the case is closed by superimposing lid 1 on tray 2, it is possible to stack several cases on one another (see FIGS. 7 and 8) without damaging the epidermis or skin grafts contained therein.

Finally, according to a further peculiar characteristic of the finding, both lid 1 and tray 2 of the case have a peripheral flange F which is heat sealed peripherally so as to seal hermetically the case in which the cultivated epidermis or skin graft has been placed.

The liquid and gas impermeable material forming the case is preferably transparent PET or other material which is chemically and biologically inert both at the temperature of use, particularly between −80° C. and +35° C. and at the heat sealing temperature.

The present invention has been described and illustrated according to preferred embodiments thereof, however, it should be understood that those skilled in the art can make equivalent modifications and/or replacements without departing from the scope of the present industrial invention.

1. A case for the protection and the transportation of epidermis or skin grafts, characterized in that it is made of a liquid and gas impermeable, transparent material which is chemically and biologically inert, and includes a lid (1) and a tray (2) able to be superimposed and fitted on each other so as to define a closed, essentially flat space (V) able to contain the epidermis or skin graft immersed in a preservation liquid under stretched conditions, both said lid (1) and said tray (2) being provided with a peripheral flange (F) which is able to be heat sealed.

2. The case according to claim 1, characterized in that said space (V) is small enough to avoid the wrinkling of the skin graft contained therein, even if the case is inclined.

3. The case according to the preceding claims, characterized in that lid (1) has an essentially flat central portion which is lowered with respect to its peripheral edge and is provided with a continues peripheral little channel which projects from the underlying surface so as to form a corresponding peripheral lower rib (3) projecting from the flat surface of the lowered portion of the lid.

4. The case according to claim 3, characterized in that tray (2) has a flat bottom surrounded and delimited by a continuous peripheral rib (4) which is raised with respect to the bottom and outside which there is provided a small channel (5) running parallel to said rib (4) and able to fit said lower rib (3) of lid (1).

5. The case according to the preceding claims, characterized in that the bottom of tray (2) is deeper than the peripheral small channel (5).

6. The case according to the preceding claims, characterized in that space (V) able to receive the cultivated epidermis or skin graft and the culture liquid necessary to its preservation is defined by the empty space delimited at the lower side by the central portion of tray (2) and at the upper side by the flat bottom of lid (1).

7. The case according to the preceding claims, characterized in that said lid (1) and said tray (2) are superimposed by fitting rib (3) into small channel (5), the liquid and gas sealing being ensured by that the fitting portions of lid (1) and tray (2) are shaped such as to provide perfectly conjugated surfaces.

8. The case according to the preceding claims, characterized in that once the case is closed by superimposing lid (1) on tray (2) it is possible to stack several cases on one another without damaging the epidermis or skin grafts contained therein.

9. The case according to the preceding claims, characterized in that said liquid and gas impermeable, transparent material which is chemically and biologically inert keeps such characteristics both at the temperature of use and at the heat sealing temperature and is formed by PET or the like.
